(19) United States
(12) Patent Application Publication
(10) Pub. No.: US 2002/0182599 A1
(43) Pub. Date: Dec. 5, 2002

(54) Methods for diagnosing and treating heart disease

(76) Inventor: Mark C. Fishman, Newton Center, MA (US)

Correspondence Address: Karen L. Elbing, Ph.D., Clark & Elbing LLP, 176 Federal Street, Boston, MA, 02110, US

(21) Appl. No.: 09/759,508

(22) Filed: Jan. 12, 2001

Related U.S. Application Data
(60) Provisional application No. 60/175,787, filed on Jan. 12, 2000.

(51) Int. Cl. C12Q 1/68; C12P 19/34

(52) U.S. Cl. 435/6; 435/91.2

(57) ABSTRACT

The invention provides methods of diagnosing heart disease, such as heart failure, screening methods for identifying compounds that can be used to treat or to prevent heart disease, and methods of using these compounds to treat or to prevent heart disease. The invention also provides animal model systems for carrying out the screening methods.

### CROSS REFERENCE TO RELATED APPLICATIONS

This application claims priority from U.S. provisional patent application 60/175,786, filed Jan. 12, 2000.

### FIELD OF THE INVENTION

This invention relates to methods for diagnosing and treating heart disease.

### BACKGROUND OF THE INVENTION

Heart disease is a general term used to describe many different heart conditions. For example, coronary artery disease, which is the most common heart disease, is characterized by constriction or narrowing of the arteries supplying the heart with oxygen-rich blood, and can lead to myocardial infarction, which is the death of a portion of the heart muscle. Heart failure is a condition resulting from the inability of the heart to pump an adequate amount of blood through the body. Heart failure is not a sudden, abrupt stop of heart activity, but, rather, typically develops slowly over many years, as the heart gradually loses its ability to pump blood efficiently. Risk factors for heart failure include coronary artery disease, hypertension, valvular heart disease, cardiomyopathy, disease of the heart muscle, obesity, diabetes, and a family history of heart failure.

### SUMMARY OF THE INVENTION

The invention provides diagnostic, drug screening, and therapeutic methods based on the observation that mutation of the titin gene leads to a phenotype in zebrafish that is similar to mammalian heart failure.

In one aspect, the invention provides a method of determining whether a test subject (e.g., a mammal, such as a human) has, or is at risk of developing, a titin-related disease or condition (e.g., heart failure). This method involves analyzing a nucleic acid molecule of a sample from the test subject to determine whether the test subject has a mutation (for example, a mutation in a cardiac-specific exon, such as the N2B exon; e.g., the pickwick mutation; see below) in a titin gene. The presence of such a mutation is an indication that the test subject has, or is at risk of developing, a titin-related disease. This method can further involve using nucleic acid molecule primers specific for the titin gene for nucleic acid molecule amplification of the titin gene by the polymerase chain reaction, or sequencing titin nucleic acid molecules from the test subject.

In another aspect, the invention provides a screening method for identifying a compound that can be used to treat or to prevent heart failure. This method involves contacting an organism (e.g., a zebrafish) having a titin mutation (for example, a mutation in a cardiac-specific exon, such as the N2B exon; e.g., the pickwick mutation) and a phenotype characteristic of heart failure with the compound, and determining the effect of the compound on the phenotype. Detection of an improvement in the phenotype indicates the identification of a compound that can be used to treat or to prevent heart failure.

In another aspect, the invention provides a method of treating or preventing heart disease, such as heart failure, in a patient. This method involves administering to the patient a compound identified using the screening method described above. A patient treated using this method can have a mutation in the titin gene.

In a further aspect, the invention provides a non-human animal (e.g., a zebrafish or a mouse) that has a mutation in a titin gene. The mutation can be, for example, in a cardiac-specific exon of the titin gene, such as the N2B exon, and can result in production of a truncated titin product, for example, by the introduction of a stop codon.

By “polypeptide” or “polypeptide fragment” is meant a chain of two or more amino acids, regardless of any post-translational modification (e.g., glycosylation or phosphorylation), constituting all or part of a naturally or non-naturally occurring polypeptide. By “post-translational modification” is meant any change to a polypeptide or polypeptide fragment during or after synthesis. Post-translational modifications can be produced naturally (such as during synthesis within a cell) or generated artificially (such as by recombinant or chemical means). A “protein” can be made up of one or more polypeptides.

By “titin,” “titin protein,” or “titin polypeptide” is meant a polypeptide that has at least 45%, preferably at least 60%, more preferably at least 75%, and most preferably at least 90% amino acid sequence identity to the sequence of the human (see below) or the zebrafish titin polypeptides. Polypeptide products from splice variants of titin gene sequences and titin genes containing mutations are also included in this definition. A titin polypeptide as defined herein plays a role in heart development, modeling, structure, and contractility. It can be used as a marker of heart disease, such as heart failure.

By a “titin nucleic acid molecule” is meant a nucleic acid molecule, such as a genomic DNA, cDNA, or RNA (e.g., mRNA) molecule, that encodes titin, a titin protein, a titin polypeptide, or a portion thereof, as defined above.

The term “identity” is used herein to describe the relationship of the sequence of a particular nucleic acid molecule or polypeptide to the sequence of a reference molecule of the same type. For example, if a polypeptide or nucleic acid molecule has the same amino acid or nucleotide residue at a given position, compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications. Conservative substitutions typically include substitutions within the following groups: glycine, alanine, valine, isoleucine, and leucine; aspartic acid, glutamic acid, asparagine, and glutamine; serine and threonine; lysine and arginine; and phenylalanine and tyrosine.

A nucleic acid molecule or polypeptide is said to be “substantially identical” to a reference molecule if it exhibits, over its entire length, at least 51%, preferably at least 55%, 60%, or 65%, and most preferably 75%, 85%, 90%, or 95% identity to the sequence of the reference molecule. For polypeptides, the length of comparison sequences is at least 16 amino acids, preferably at least 20 amino acids, more preferably at least 25 amino acids, and most preferably at least 35 amino acids. For nucleic acid molecules, the length of comparison sequences is at least 50 nucleotides, preferably at least 60 nucleotides, more preferably at least 75 nucleotides, and most preferably at least 110 nucleotides.

A titin nucleic acid molecule or titin polypeptide is “analyzed” or subject to “analysis” if a test procedure is carried out on it that allows the determination of its biological activity or whether it is wild type or mutated. For example, one can analyze the titin genes of an animal (e.g., a human or a zebrafish) by amplifying genomic DNA of the animal using the polymerase chain reaction, and then determining whether the amplified DNA contains a mutation, for example, the pickwick mutation, by, e.g., nucleotide sequence or restriction fragment analysis.

By “probe” or “primer” is meant a single-stranded DNA or RNA molecule of defined sequence that can base pair to a second DNA or RNA molecule that contains a complementary sequence (“target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes or primers specific for titin nucleic acid molecules, preferably, have greater than 45% sequence identity, more preferably at least 55-75% sequence identity, still more preferably at least 75-85% sequence identity, yet more preferably at least 85-99% sequence identity, and most preferably 100% sequence identity to the sequences of human (see below) or zebrafish titin.

Probes can be detectably-labeled, either radioactively or non-radioactively, by methods that are well-known to those skilled in the art. Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), and other methods that are well known to those skilled in the art.

A molecule, e.g., an oligonucleotide probe or primer, a gene or fragment thereof, a cDNA molecule, a polypeptide, or an antibody, can be said to be “detectably-labeled” if it is marked in such a way that its presence can be directly identified in a sample. Methods for detectably-labeling molecules are well known in the art and include, without limitation, radioactive labeling (e.g., with an isotope, such as ³²P or ³⁵S) and nonradioactive labeling (e.g., with a fluorescent label, such as fluorescein).

By a “substantially pure polypeptide” is meant a polypeptide (or a fragment thereof) that has been separated from proteins and organic molecules that naturally accompany it. Typically, a polypeptide is substantially pure when it is at least 60%, by weight, free from the proteins and naturally-occurring organic molecules with which it is naturally associated. Preferably, the polypeptide is a titin polypeptide that is at least 75%, more preferably at least 90%, and most preferably at least 99%, by weight, pure. A substantially pure titin polypeptide can be obtained, for example, by extraction from a natural source (e.g., isolated heart tissue), by expression of a recombinant nucleic acid molecule encoding a titin polypeptide, or by chemical synthesis. Purity can be measured by any appropriate method, e.g., by column chromatography, polyacrylamide gel electrophoresis, or HPLC analysis.

A polypeptide is substantially free of naturally associated components when it is separated from those proteins and organic molecules that accompany it in its natural state. Thus, a protein that is chemically synthesized or produced in a cellular system different from the cell in which it is naturally produced is substantially free from its naturally associated components. Accordingly, substantially pure polypeptides not only include those derived from eukaryotic organisms, but also those synthesized in E. coli or other prokaryotes.

An antibody is said to “specifically bind” to a polypeptide if it recognizes and binds to the polypeptide (e.g., a titin polypeptide), but does not substantially recognize and bind to other molecules (e.g., non-titin related polypeptides) in a sample, e.g., a biological sample that naturally includes the polypeptide.

By “high stringency conditions” is meant conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8× SSC, 0.2 M Tris-Cl, pH 7.6, 1× Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing and 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

By “sample” is meant a tissue biopsy, amniotic fluid, cell, blood, serum, urine, stool, or other specimen obtained from a patient or test subject. The sample can be analyzed to detect a mutation in a titin gene, or expression levels of a titin gene, by methods that are known in the art. For example, methods such as sequencing, single-strand conformational polymorphism (SSCP) analysis, or restriction fragment length polymorphism (RFLP) analysis of PCR products derived from a patient sample can be used to detect a mutation in a titin gene; ELISA can be used to measure levels of titin polypeptide; and PCR can be used to measure the level of a titin nucleic acid molecule.

By “titin-related disease” or “titin-related condition” is meant a disease or condition that results from inappropriately high or low expression of a titin gene, or a mutation in a titin gene that alters the biological activity of a titin nucleic acid molecule or polypeptide. Titin-related diseases and conditions can arise in any tissue in which titin is expressed during prenatal or post-natal life. Titin-related diseases and conditions can include heart diseases, such as heart failure. Specific examples of different types of heart failure are provided below.

The invention provides several advantages. For example, using the diagnostic methods of the invention, it is possible to detect an increased likelihood of heart disease, such as heart failure, in a patient, so that appropriate intervention can be instituted before any symptoms occur. This may be useful, for example, with patients in high-risk groups for heart failure (see above). Also, the diagnostic methods of the invention facilitate determination of the etiology of an existing heart condition, such as heart failure, in a patient, so that an appropriate approach to treatment can be selected. In addition, the screening methods of the invention can be used to identify compounds that can be used to treat or to prevent heart conditions, such as heart failure.

Other features and advantages of the invention will be apparent from the following detailed description and the claims.

### BRIEF DESCRIPTION OF THE DRAWING

FIG. 1 is a schematic representation of the domain structure of the human titin filament. The nucleotide and amino acid sequences of human titin are provided in SEQ ID NOs:1 and 2, respectively. The modular architecture of cardiac titin as predicted by its full-length cDNA is shown.

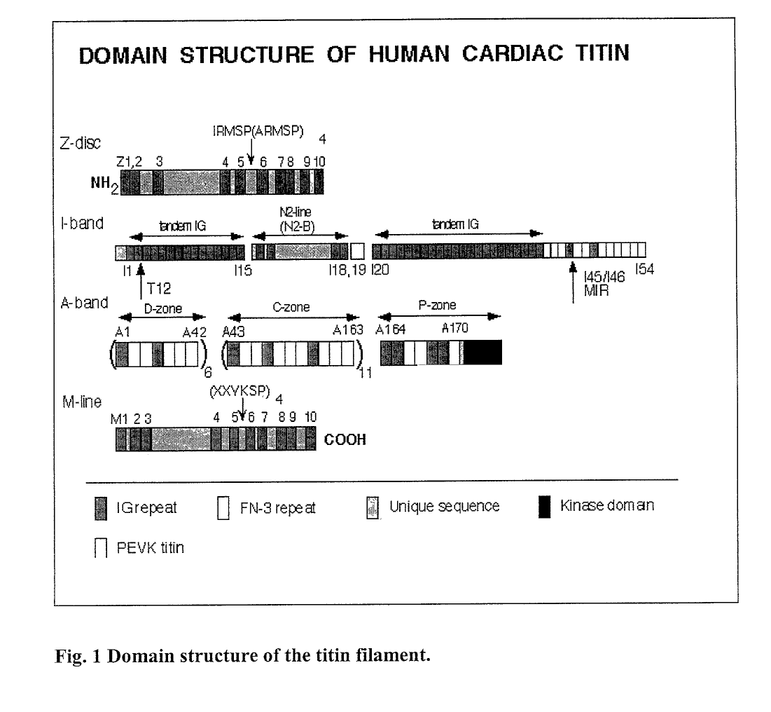

A total of 244 copies of 100 residue repeats, as indicated by vertical rectangles, are contained in the molecule. One hundred and twelve of these belong to the Ig domain, and 132 belong to the FN3 superfamily. The titin kinase domain, as well as the PEVK element N2-B 163-residue variant are also shown. Within the A-band, the D-zone contains six tandem repeats of the seven domains shown (A1 through A42), and the C-zone contains 11 tandem repeats of the 11 domains shown (A43 through Al 63). The positions of the tandemly repeated RMSP and VKSP motifs in the Z-disc and M-line region are also shown

### DETAILED DESCRIPTION

The invention provides methods of diagnosing heart disease, screening methods for identifying compounds that can be used to treat or to prevent heart disease, and methods of treating or preventing heart disease using these compounds. The invention also provides animal model systems that can be used in the screening methods of the invention.

In particular, we have discovered that a mutation (the pickwick mutation) in the titin gene is associated with heart disease, such as heart failure. Titin, which is also known as “connectin,” is the largest known single-chain protein, having a molecular weight of about 3,000 kDa. Titin is a structural protein, and plays a central role in the assembly and elasticity of vertebrate skeletal and cardiac muscle. Thus, the diagnostic methods of the invention involve detection of mutations in the titin gene, while the compound identification methods of the invention involve screening for compounds that affect the phenotype of titin mutants or other models of heart disease, such as heart failure. Compounds identified in this manner can be used in methods to treat or to prevent heart disease (e.g., heart failure). The diagnostic, screening, and therapeutic methods of the invention, as well as the animal model systems of the invention, are described further, as follows.

Diagnostic Methods

Titin nucleic acid molecules, polypeptides, and antibodies can be used in methods to diagnose or to monitor diseases and conditions involving mutations in, or inappropriate expression of, titin genes. As discussed further below, the pickwick mutation in zebrafish, which is present in the titin gene, is characterized by a phenotype that is similar to that of heart failure in humans. Thus, detection of abnormalities in titin genes or their expression can be used in methods to diagnose, or to monitor treatment or development of, human heart disease, such as heart failure. For use as references, the human cardiac titin cDNA sequence can be found at: http://www.embl-heidelberg.de/Extemallnfo/Titin/cardiacseq.html (SEQ ID NO:1), while the corresponding protein sequence can be found at: http://www.embl-heidelberg.de/Extemallnfo/Titin/cardiacpep.html (SEQ ID NO:2).

As noted above, the diagnostic methods of the invention can be used, for example, with patients that have heart failure, in an effort to determine its etiology and, thus, to facilitate selection of an appropriate course of treatment. The diagnostic methods can also be used with patients that have not yet developed heart failure, but who are at risk of developing such a disease, or with patients that are at an early stage of developing such a disease. Also, the diagnostic methods of the invention can be used in prenatal genetic screening, for example, to identify parents who may be carriers of a recessive titin mutation.

Examples of heart failure that can be diagnosed (and treated) using the methods of the invention include congestive heart failure, which is characterized by fluid in the lungs or body, resulting from failure of the heart in acting as a pump; right sided heart failure (right ventricular), which is characterized by failure of the pumping action of the right ventricle, resulting in swelling of the body, especially the legs and abdomen; left sided heart failure (left ventricular), which is caused by failure of the pumping action of the left side of the heart, resulting in congestion of the lungs; forward heart failure, which is characterized by the inability of the heart to pump blood forward at a sufficient rate to meet the oxygen needs of the body at rest or during exercise; backward heart failure, which is characterized by the ability of the heart to meet the needs of the body only if heart filling pressures are abnormally high; low-output, which is characterized by failure to maintain blood output; and high-output, which is characterized by heart failure symptoms, even when cardiac output is high.

Titin may also play a role in cardiovascular diseases other than heart failure, such as coronary artery disease or conditions associated with valve formation defects, and, thus, detection of abnormalities in titin genes or their expression can be used in methods to diagnose and monitor these conditions as well. The methods of the invention can be used to diagnose (or to treat) the disorders described herein in any mammal, for example, humans, domestic pets, or livestock.

Titin abnormalities that can be detected using the diagnostic methods of the invention include those characterized by, for example, (i) abnormal titin polypeptides, (ii) titin genes containing mutations that result in the production of such polypeptides, and (iii) titin mutations that result in production of abnormal amounts of titin. Detection of such abnormalities, thus, can be used in methods to diagnose human heart disease, such as heart failure. Exemplary of the titin mutations that can be detected using the methods of the invention is the pickwick mutation (see below).

Detection of titin mutations can be carried out using any diagnostic technique. For example, a biological sample obtained from a patient can be analyzed for one or more mutations in titin nucleic acid molecules (e.g., the pickwick mutation) using a mismatch detection approach. Generally, this approach involves polymerase chain reaction (PCR) amplification of nucleic acid molecules from a patient sample, followed by identification of a mutation (i.e., a mismatch) by detection of altered hybridization, aberrant electrophoretic gel migration, binding, or cleavage mediated by mismatch binding proteins, or by direct nucleic acid molecule sequencing. Any of these techniques can be used to facilitate detection of mutant titin genes, and each is well known in the art. Examples of these techniques are described by Orita et al. (Proc. Natl. Acad. Sci. U.S.A. 86:2766-2770, 1989) and Sheffield et al. (Proc. Natl. Acad. Sci. U.S.A. 86:232-236, 1989).

Mutation detection assays also provide an opportunity to diagnose a titin-mediated predisposition to heart disease before the onset of symptoms. For example, a patient heterozygous for a titin mutation that suppresses normal titin biological activity or expression may show no clinical symptoms of a titin-related disease, and yet possess a higher than normal probability of developing heart disease, such as heart failure. Given such a diagnosis, a patient can take precautions to minimize exposure to adverse environmental factors, and can carefully monitor their medical condition, for example, through frequent physical examinations. As mentioned above, this type of diagnostic approach can also be used to detect titin mutations in prenatal screens.

The titin diagnostic assays described above can be carried out using any biological sample (for example, a muscle tissue sample) in which titin is normally expressed. Because of the limited number of tissues in which titin is expressed, as well as the relative difficulties involved in obtaining samples of these tissues, it may be preferable to detect mutant titin genes in another, more easily obtained sample type, such as blood or amniotic fluid samples using, for example, mismatch detection techniques. Preferably, the DNA in such a sample is subjected to PCR amplification prior to analysis.

Levels of titin expression in a patient sample can be determined by using any of a number of standard techniques that are well known in the art. For example, titin expression in a biological sample (e.g., a blood or tissue sample, or amniotic fluid) from a patient can be monitored by standard northern blot analysis or by quantitative PCR (see, e.g., Ausubel et al., supra; PCR Technology: Principles and Applications for DNA Amplification, H. A. Ehrlich, Ed., Stockton Press, NY; Yap et al., Nucl. Acids. Res. 19:4294, 1991).

In yet another diagnostic approach of the invention, an immunoassay is used to detect or to monitor titin protein levels in a biological sample. Titin-specific polyclonal or monoclonal antibodies can be used in any standard immunoassay format (e.g., ELISA, Western blot, or RIA; see, e.g., Ausubel et al., supra) to measure titin polypeptide levels. These levels are compared to wild-type titin levels. For example, a decrease in titin production may be indicative of a condition or a predisposition to a condition involving insufficient titin biological activity.

Immunohistochemical techniques can also be utilized for titin detection. For example, a tissue sample can be obtained from a patient, sectioned, and stained for the presence of titin using an anti-titin antibody and any standard detection system (e.g., one that includes a secondary antibody conjugated to horseradish peroxidase). General guidance regarding such techniques can be found in, e.g., Bancroft et al., Theory and Practice of Histological Techniques, Churchill Livingstone, 1982, and in Ausubel et al., supra.

Identification of Molecules That Can Be Used to Treat or to Prevent Heart Failure

Identification of a mutation in titin as resulting in a phenotype that is related to heart failure facilitates the identification of molecules (e.g., small organic molecules, peptides, or nucleic acid molecules) that can be used to treat or to prevent heart failure. The effects of candidate compounds on heart failure can be investigated using, for example, the zebrafish system. The zebrafish, Danio rerio, is a convenient organism to use in genetic analysis of vascular development. In addition to its short generation time and fecundity, it has an accessible and transparent embryo, allowing direct observation of blood vessel function from the earliest stages. As discussed further below, zebrafish and other animals having mutations in the titin gene, which can be used in these methods, are also included in the invention.

In one example of the screening methods of the invention, a zebrafish having a mutation in the titin gene (e.g., a zebrafish having the pickwick mutation; see below) is contacted with a candidate compound, and the effect of the compound on the development of a heart abnormality that is characteristic of heart failure, or on the status of such an existing heart abnormality, is monitored, relative to an untreated, identically mutant control. As discussed further below, zebrafish having the pickwick mutation are characterized by, for example, reduction of peak systolic pressures, stretched and thin myocardium, excess cardiac jelly, absent A-V cushions, and an obstructed ventricular outflow tract. Thus, these characteristics (in addition to other characteristics of heart failure) can be monitored using the screening methods of the invention.

After a compound has been shown to have a desired effect in the zebrafish system, it can be tested in other models of heart disease, for example, in mice or other animals having a mutation in the titin gene. Alternatively, testing in such animal model systems can be carried out in the absence of zebrafish testing.

Candidate compounds can be purified (or substantially purified) molecules or can be one component of a mixture of compounds (e.g., an extract or supernatant obtained from cells; Ausubel et al., supra). In a mixed compound assay, the effect on a phenotype of heart failure is tested against progressively smaller subsets of the candidate compound pool (e.g., produced by standard purification techniques, e.g., HPLC or FPLC) until a single compound or minimal compound mixture is demonstrated to have the desired effect.

Test compounds that can be screened in the methods described above can be chemicals that are naturally occurring or artificially derived. Such compounds can include, for example, polypeptides, synthesized organic molecules, naturally occurring organic molecules, nucleic acid molecules, and components thereof Candidate compound can be found, for example, in a cell extract, mammalian serum, or growth medium in which mammalian cells have been cultured.

In general, novel drugs for prevention or treatment of mutant titin-related heart diseases can be identified from large libraries of both natural products, synthetic (or semi-synthetic) extracts, and chemical libraries using methods that are well known in the art. Those skilled in the field of drug discovery and development will understand that the precise source of test extracts or compounds is not critical to the screening methods of the invention. Accordingly, virtually any number of chemical extracts or compounds can be screened using these methods. Examples of such extracts or compounds include, but are not limited to, plant-, fungal-, prokaryotic-, or animal-based extracts, fermentation broths, and synthetic compounds, as well as modifications of existing compounds.

Numerous methods are also available for generating random or directed synthesis (e.g., semi-synthesis or total synthesis) of any number of chemical compounds, including, but not limited to, saccharide-, lipid-, peptide-, and nucleic acid molecule-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). In addition, natural and synthetically produced libraries can be produced, if desired, according to methods known in the art, e.g., by standard extraction and fractionation. Furthermore, if desired, any library or compound can be readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their therapeutic activities for heart failure can be employed whenever possible.

When a crude extract is found to have an effect on the development or persistence of heart failure, further fractionation of the positive lead extract can be carried out to isolate chemical constituents responsible for the observed effect. Thus, the goal of the extraction, fractionation, and purification process is the careful characterization and identification of a chemical entity within the crude extract having a desired activity. The same assays described herein for the detection of activities in mixtures of compounds can be used to purify the active component and to test derivatives of these compounds. Methods of fractionation and purification of such heterogeneous extracts are well known in the art. If desired, compounds shown to be useful agents for treatment can be chemically modified according to methods known in the art.

Treatment or Prevention of Heart Failure

Compounds identified using the screening methods described above can be used to treat patients that have or are at risk of developing heart disease, such as heart failure. Such treatment may be required only for a short period of time, or may, in some form, be required throughout a patient's lifetime. Any continued need for treatment, however, can be determined using, for example, the diagnostic methods described above. In considering various therapies, it is understood that such therapies are, preferably, targeted to the affected or potentially affected organ, that is, the heart.

Treatment or prevention of diseases resulting from a mutated titin gene can be accomplished, for example, by modulating the function of a mutant titin protein, delivering normal titin protein to the appropriate cells, altering the levels of normal or mutant titin protein, replacing a mutant titin gene with a normal titin gene or, administering a normal titin gene. It is also possible to correct a titin defect by modifying the physiological pathway (e.g., a signal transduction pathway) in which the titin protein participates.

In a patient diagnosed as heterozygous for a titin mutation, or as susceptible to titin mutations or aberrant titin expression (even if those mutations or expression patterns do not yet result in alterations in titin expression or biological activity), any of the above-described therapies can be administered before the occurrence of the disease phenotype. In particular, compounds shown to modulate titin expression or to have an effect on the phenotype of titin mutants can be administered to patients diagnosed with potential or actual heart disease by any standard dosage and route of administration.

Any appropriate route of administration can be employed to administer a compound found to be effective in treating or preventing heart failure, according to the invention. For example, administration can be parenteral, intravenous, intra-arterial, subcutaneous, intramuscular, intraventricular, intracapsular, intraspinal, intracisternal, intraperitoneal, intranasal, aerosol, or oral. However, as noted above, preferably, the administration is local to the afflicted tissue, that is, the heart. Therapeutic formulations can be in the form of liquid solutions or suspensions; for oral administration, formulations can be in the form of tablets or capsules; and for intranasal formulations, in the form of powders, nasal drops, or aerosols.

A therapeutic compound of the invention can be administered within a pharmaceutically acceptable diluent, carrier, or excipient, in unit dosage form. Administration can begin before or after the patient is symptomatic. Methods that are well known in the art for making formulations are found, for example, in Remington's Pharmaceutical Sciences, (18^{th} edition), ed. A. Gennaro, 1990, Mack Publishing Company, Easton, Pa. Formulations for parenteral administration can, for example, contain excipients; sterile water; or saline; polyalkylene glycols, such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Other potentially useful parenteral delivery systems for compounds identified using the methods of the invention include ethylene-vinyl acetate copolymer particles, osmotic pumps, implantable infusion systems, and liposomes. Formulations for inhalation can contain excipients, for example, lactose, or can be aqueous solutions containing, for example, polyoxyethylene-9-lauryl ether, glycocholate, and deoxycholate, or can be oily solutions for administration in the form of nasal drops, or as a gel.

Titin nucleic acid molecules and polypeptides can also be used in tissue engineering, for example, in the manufacture of artificial or partially artificial hearts. As mentioned above, titin plays a role in cardiovascular elasticity, integrity, and contractility. Thus, a titin nucleic acid molecule or polypeptide, as described above, can be used to impart such characteristics on an artificial or partially artificial heart.

Animal Model Systems

The invention also provides animal model systems for use in carrying out the screening methods described above. Examples of these model systems include zebrafish and other animals, such as mice, that have mutations in a titin gene. For example, a zebrafish model that can be used in the invention can include a mutation that results in a lack of titin production or production of a truncated (e.g., by introduction of a stop codon) or otherwise altered titin gene product. The mutation can, for example, result in the presence of a stop codon in a cardiac-specific exon, such as the N2B exon (e.g., in the IS3 region; see below). The mutation can be in a region encoding the I band, resulting in the production of a protein in which the I band is truncated and the A band and M line are absent, or can be in a region encoding another portion of the molecule, such as the A band or M line region. As a specific example, a zebrafish having the pickwick mutation can be used.

Experimental Results

During a large-scale mutagenesis screening of the zebrafish genome, a group of mutations were identified that affect cardiac contractility. One of these mutations, called pickwick (pik), dramatically reduces the function of both chambers, causing a recessive, lethal form of heart failure in the zebrafish embryo.

Direct in vivo recording of ventricular pressures by the null balance feedback system shows that pickwick causes a 5.8 fold reduction of peak systolic pressures, compared to age-matched controls (0.084+/−0.008 vs. 0.49+/−0.06 mm Hg). Morphological analysis of pickwick revealed a stretched and thin myocardium, excess cardiac jelly, absent A-V cushions, and an obstructed ventricular outflow tract. Extensive ultrastructural defects were found by transmission electron microscopy, affecting the assembly of Z-discs and the organization of myofilaments. Reciprocal blastomere transplants identified pickwick as a cell autonomously acting mutation of the myoblast lineage.

A positional cloning approach was adopted for gene identification. The pickwick mutation has been assigned to a small chromosomal interval, which is covered by BAC clones. The titin gene spans this interval, and thus the pickwick phenotype is due to a mutation in the titin gene. In particular, the pickwick gene was mapped to linkage group (LG) 9 by bulk segregant analysis using the pikm242 allele, which is one of five cardiac specific alleles (pikm242, pikm171, pikm740, pikm186,pikmnm2). A panel of Z-markers in the linkage group was tested for simple sequence length polymorphisms (SSLPs) using 931 homozygous mutant embryos. Markers Z8363 and Z26463 were shown to flank the pickwick locus, defining a 1.2 cM interval containing the gene. It is estimated that 1 cM corresponds to 500-600 kb DNA in the zebrafish genome (Postlethwait et al., Science 264:699-703, 1994). We thus initiated chromosomal walking from the Z8363 marker, which is 0.7 cM from the mutation (12 recombinants out of 1750 meioses).

A positive YAC clone (YAC5) was identified that had a T7 end that is highly homologous to human titin coding sequences. As the titin genomic region was estimated to be over 300 kb in humans, we decided to identify BAC clones based on sequence information of the zebrafish titin EST clones. A physical contig was constructed based on these sequences, which covers the whole titin genomic area. Single stranded conformational polymorphisms (SSCPs) were developed from the ends and internal sequences of these clones were used for fine recombinational mapping. One SSCP marker inside the titin genomic area (B9F2) picked up one recombinant from the Z26463 side. The other four SSCP markers inside the titin genomic area (B4SP1, B2T7, B7SP, and B6SP) picked up zero recombinants out of 1750 meioses. These genetic data indicated that the pickwick locus is very close to or within the titin genomic region.

As the titin cDNA alone is around 82 kb, rescuing the phenotype by RNA injection could prove to be quite difficult. However, we found evidence that the pickwick locus is within the titin gene by identification of point mutations in one of the pickwick alleles. As most of the alleles of pickwick have a cardiac-specific phenotype, we presumed that the point mutation is located in a cardiac-specific exon. We focused on the N2B domain in the I-band of titin, as all of the cardiac isoforms are N2B domain based. The zebrafish N2B domain was cloned by RT-PCR. It is a 4.3 kb cDNA encoding infrastructures similar to those in humans and mice, and contains 4 IG repeats and three unique sequences, including longer IS3 and shorter IS1.

N2B domains from pickwick mutant embryos were then cloned. RNA mismatch analysis was performed to identify the location of the point mutation. One mismatch between the PCR products from pikm171 and pikm242 was identified. Sequencing of the PCR product resulted in the identification of a T→G transition in the pikm171 allele. This mutation resulted in a change of leucine in the IS3 fragment of N2B domain (N2B-IS3) into a stop codon. The mutation was confirmed in all of the seven homozygous pikm171 mutant embryos, but none of the four pikm242 homozygous embryos. A truncated version of titin is predicted to be in the pikm171 mutant, only as a cardiac specific isoform. It should contain the Z-disc and part of the I-band and be sized around 4,000 amino acids, based on comparison to the homologous human titin sequence, which has a full length of 27,000 amino acids. The identification of a non-sense mutation in the cardiac specific N2B domain in pikm171 allele confirmed the hypothesis that titin is the pickwick gene.

Titin was expressed in the zebrafish embryo during the period when the pickwick phenotype was first detected. Whole mount in situ hybridization analysis indicated that titin was expressed strongly in both the heart and the somites at 24 hpf. Titin mRNA expression in the heart is normal in pikm171. We confirmed the notion that N2B is a cardiac-specific exon in zebrafish by labeling a probe in the IS3 domain for the whole mount in situ hybridization.

The identification of a point mutation in the N2B domain thus establishes pickwick as the first in vivo vertebrate system to study the functions of titin in the heart. If titin functions as a spring, as proposed, it is expected that the contraction will be much weaker. This is exactly what we observed in pickwick mutant embryos. If titin functions as a template during the sarcomere assembly, a “silent heart” phenotype would be expected in titin null mutation. According to the current model of the myofibrillogenesis (Dabiri et al., Proc. Natl. Acad. Sci. U.S.A. 94:9493-9498, 1997), the thick element and/or the sarcomere could not assemble into a beating machine. In contrast, the hearts in the homozygous pikm171 mutant embryos and all of the other pickwick alleles still beat, despite being weaker.

The mutation in pikm171 predicts a truncated protein in which most of the elastic I-band is deleted and the C-terminal A-band and M-line regions eliminated. It thus could be considered as a null mutation in terms of function as a spring and a potential dominant negative mutation in terms of its function as a template for sarcomere assembly (Turnacioglu et al., Mol. Biol. Cell 8:705-717, 1997). The observation of the weak beating in the pickwick mutant embryos suggested the existence of primary contractile machinery without titin. Indeed, thick and thin elements can be detected in the ventricular myocardium cells. They have the capacity to assemble into a functional beating structure in the absence of titin.

We thus have carried out a detailed physiological and morphological analysis of pickwick, a zebrafish heart function mutation that reduces the contractility of both chambers. Several pieces of evidence pointed out that titin is the pickwick gene. Genetic analysis linked the pickwick locus closely to the titin genomic area. The identification of the pikmVO62H, a pickwick allele that has an additional somite phenotype, can be explained by the titin hypothesis. The point mutation is expected to be in the common exons that are shared between the cardiac and somatic isoforms of titin. Evidence confirming this hypothesis came from the identification of a point mutation in the cardiac specific N2B domain of titin in one of the pickwick alleles, pikm171. We went on to show that sarcomere structure is disrupted in the myocardium cells of pikm171, but not the somatic muscle cells. The expression pattern of titin is consistent with this phenotype. Strong expression in both cardiac and skeletal muscles was detected at the onset of the pickwick phenotype.

Thus, our observation in zebrafish is in consistent with the notion that titin functions as a spring during the muscle contraction. As titin is a sarcomere structure protein, it is conceivable that myocardium is affected cell-autonomously in pickwick mutant embryos. The thin and stretched morphology could be due to the mechanic tension generated from the failure to form higher-order sarcomere structure and the loss of spring. The mechanic tension may also be the reason for the separation between the myocardium cells and endocardium cells, generating the excess cardiac jelly. However, there is a possibility that the differentiation program of the myocardium was affected in the titin mutation. The valve formation phenotype in pickwick mutant embryos could be a secondary defect. It has been suggested that the process of endothelial invasion during valve formation is under control of a localized myocardial signal. (For a review, see Fishman et al., Development 124:2099-2117, 1997.) The physical distance between myocardium and endocardium and/or the stalked differential program in the myocardium could be the reason that prevents a normal cushion formation.

Material and Methods

Zebrafish Strains and Maintenance

Zebrafish were maintained and staged as described. pikm242, pikm171, pikm740, pikm186 were generated in a screen on the AB background (Stainier et al., Development 123:285-292, 1996). pikmVO62H and pikmnml2 were generated in a screen on the TL background. Mapping strains were constructed by crossing pikm242 into india strain. pikm171 embryos used in expression analysis and EM were obtained from pair wise matings of pikm171/TL heterozygotes.

In situ Hybridization

Whole mount in situ hybridization was performed as described (Thisse et al., Development 119:1203-1215, 1993). T5 probe was generated by digestion of the EST clone A1629069 (Research Genetics). The N2B and N2A probe were generated through PCR with a tagged T7 promoter. The primer pairs are:

| | | |
|---|---|---|
| P238F: | 5′-AGGGACACTCAGAGACCATAG | (SEQ ID NO:3); and |
| P3785RT: | 5′-TAATACGACTCACTATAGGGGTCTGAGGATACTCGCCTTC | (SEQ ID NO:4). |

Mapping of Pickwick

Linkage was established using DNA from 16 homozygous mutations and 16 heterozygous or wild type pick pikm242/indian embryos in bulk segregation analysis (Michelmore et al., Proc. Natl. Acad. Sci. U.S.A. 88:9828-9832, 1991). Z-markers (simple sequence length polymorphisms, SSLP) were developed in this lab (Knapik et al., Nat. Genet. 18:338-343, 1998; Shimoda et al., Genomics 58:219-232, 1999). Genotyping products were resolved in 6% PAGE gels.

Chromosomal Walking

YAC and BAC clones were screened by PCR as pools of clones (Research Genetics) according to the manufacturer's instruction. YAC ends were cloned by plasmid rescue (Zhong et al., Genomics 48:136-138, 1998). Chimeric ends were determined by the RH panel (Geisler et al., Nat. Genet. 23:86-89, 1999). BAC DNA was extracted using the QIAgene kit and the end sequences were determined by direct sequencing. BLAST-X was performed to search for homologous sequence in Genebank. EST clones were generated by Washington University zebrafish EST project and obtained from Research Genetics. Oligonucleotides derived from the end sequences of YAC and BAC clones were used in standard PCR reactions to determine clone overlap. Primer pair B9F2 was derived from a fragment that was generated by digesting BAC5 with BamHI and then subcloning into the pUC19 vector. This clone can be hit by a primer pair derived form the T7 end of BAC7. Single-strand conformation polymorphisms (SSCP) were tested on 6% MDE acrylamide (FMC Bioproducts) gels at 40° C.

Cloning of the Zebrafish N2B Domain

Long RT-PCR was performed to amplify the N2B domain from adult zebrafish heart mRNA extracts. mRNA was extracted from pools of ten zebrafish adult hearts using TRIzole reagent (GibcolBRL), as described. The cDNA was synthesized using SuperScripTMII RNase H-Reverse Transcriptase (GibcoBRL) and then treated with RNase H. The primer is derived from EST3: I19R1: 5′-TTTGAACCACTTGAAGGTCACACCAGG (SEQ ID NO:5). Long-PCR was performed using Expand 20 kbPlus PCR System (Roch) as described. The primer pairs are:

| | |
|---|---|
| I14F1: | |
| 5′-GCTAAGAATGACTATGGAGTTGCCACAAGC | (SEQ ID NO:6) |
| I19R2: | |
| 5′-TGAACCACTTGAAGGTCACACCAGGAG | (SEQ ID NO:7) |

The 4.6 kb product was subcloned using TOPO TA Cloning Kit (Invitrogen) as described. The sequence was determined by primer walking. Forty eight reads were aligned by Phred/Phrad software to get a large contig that contains only one reading frame with around three fold coverage.

To amplify the N2B region from the homozygous mutant zebrafish embryos, three overlapping primer pairs were designed according to the adult N2B sequence. The contamination from the skeletal muscle specific titin isofoirm was thus eliminated. mRNA was extracted from pools of ten zebrafish day 2 embryos using TRIzole reagent (GibcoBRL) as described. Sequencing results indicated that the embryonic heart titin contain one less IG domain in the area.

Identification of the Point Mutation

N2B domains from the homozygous mutant embryos were further amplified by primer pairs that generate six overlapping PCR products sized between 0.6 kb and about 1 kb. RNA mismatch analysis was performed using MutationScreenerTM (Ambion) according to the manufacture's protocol. A mismatch was identified between pikm171 and pikm242 using the primer pairs:

| | | |
|---|---|---|
| P238FT: | 5′-TAATACGACTCACTATAGGGAGGGACACTCAGAGACCATAG | (SEQ ID NO:8) |
| P3785RT: | 5′-TAATACGACTCACTATAGGGGTCTGAGGATACTCGCCTTC | (SEQ ID NO:9). |

Sequencing results indicate a T→G non-sense mutation in the cDNA from homozygous pikm171 embryos. Genomic sequence in this region was amplified using the primer pair:

| | | |
|---|---|---|
| P238F: | 5′-AGGGACACTCAGAGACCATAG | (SEQ ID NO:10) |
| P341R: | 5′-GGCAATGTTACTCTCTGTTGAG | (SEQ ID NO:11) |

and sent out directly for sequencing after purification using the Geneclean Spin Kit (BIO101).

Electron Microscopy

Forty eight hour embryos were fixed overnight at 4° C. in 1.5% glutaraldehyde, 1% paraformaldehyde, 70 mM NaPO₄ pH 7.2, 3% Sucrose. They were then washed 3 times for 5 minutes each in 0.1M cacodylate buffer, pH 7.4.

Other Embodiments

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention described herein. Such equivalents are intended to be encompassed in the scope of the present invention.

All publications and patents mentioned in this specification are hereby incorporated by reference.

What is claimed is:

1. A method of determining whether a test subject has, or is at risk of developing, a titin-related disease or condition, said method comprising analyzing a nucleic acid molecule of a sample from the test subject to determine whether the test subject has a mutation in a titin gene, wherein the presence of said mutation is an indication that said test subject has, or is at risk of developing, a titin-related disease.

2. The method of claim 1, further comprising the step of using nucleic acid molecule primers specific for the titin gene for nucleic acid molecule amplification of the titin gene by the polymerase chain reaction.

3. The method of claim 1, further comprising the step of sequencing titin nucleic acid molecules from said test subject.

4. The method of claim 1, wherein said test subject is a mammal.

5. The method of claim 1, wherein said test subject is human.

6. The method of claim 1, wherein said disease or condition is heart failure.

7. The method of claim 1, wherein said mutation is the pickwick mutation.

8. A method for identifying a compound that can be used to treat or to prevent heart failure, said method comprising contacting an organism comprising a titin mutation and having a phenotype characteristic of heart failure with said compound, and determining the effect of said compound on said phenotype, wherein detection of an improvement in said phenotype indicates the identification of a compound that can be used to treat or to prevent heart failure.

9. The method of claim 8, wherein said organism is a zebrafish.

10. The method of claim 8, wherein said titin mutation is the pickwick mutation.

11. A method of treating or preventing heart failure in a patient, said method comprising administering to said patient a compound identified using the method of claim 8.

12. The method of claim 11, wherein said patient has a mutation in the titin gene.

13. The method of claim 12, wherein said mutation is the pickwick mutation.

14. A non-human animal comprising a mutation in a titin gene.

15. The non-human animal of claim 14, wherein the non-human animal is a zebrafish.

16. The non-human animal of claim 14, wherein the mutation is in a cardiac-specific exon of said titin gene.

17. The non-human animal of claim 16, wherein the mutation is in the N2B exon of said titin gene.

18. The non-human animal of claim 14, wherein the mutation results in the presence of a stop codon in said titin gene.

19. The non-human animal of claim 14, wherein the mutation is the pickwick mutation.
